(19) United States
(12) Patent Application Publication
(10) Pub. No.: US 2024/0123242 A1
(43) Pub. Date: Apr. 18, 2024

(54) AUTOMATED EXTERNAL DEFIBRILLATORS WITH MULTIPLE, MULTIFUNCTIONAL ELECTRODE PAIRS

(71) Applicant: CELLAED LIFE SAVER PTY LTD., Elanora Height (AU)

(72) Inventors: Erol TEBER, Riverwood (AU); Donovan CASEY, Riverwood (AU)

(21) Appl. No.: 18/275,593

(22) PCT Filed: Feb. 1, 2022
(86) PCT No.: PCT/IB2022/050836

§ 371 Date: Aug. 2, 2023

(30) Foreign Application Priority Data
Feb. 3, 2021 (AU) 2021900233

(51) Int. Cl. A61N 1/39; A61N 1/04

(52) U.S. Cl. CPC A61N 1/3918; A61N 1/046; A61N 1/0476; A61N 1/3904; A61N 1/3925; A61N 1/3987

(57) ABSTRACT

An automated external defibrillator (AED), comprising two pads to be placed on a patient, multiple electrode pairs provided on or between the two pads to perform multiple functions of electrical measurement and stimulation of the patient's heart, a switching circuit and a shock generation circuit connected to the multiple electrode pairs and a controller connected to the switching circuit and the shock generation circuit, wherein the controller is configured to perform spatial division multiplexing by rapidly switching between the multiple electrode pairs to automatically perform the multiple functions of electrical measurement and stimulation of the patient's heart using the multiple electrode pairs in multiple directions.

### FIELD

The present invention relates generally to automated external defibrillators (AEDs) having defibrillation pads with multiple, multifunctional electrode pairs.

### BACKGROUND

AEDs typically use a single pair of electrodes split across two defibrillation pads—one electrode per pad—to perform functions of measuring electrocardiogram (ECG) signals to detect a shockable cardiac rhythm, and delivering defibrillation shocks on a mean cardiac electrical axis.

Conventional single electrode pair AEDs suffer several shortcomings. Effective placement of the single electrode pads is critically important for the AED's electrical measurement and stimulation functions, because the signal quality of ECG signals and the efficacy of defibrillation shocks often both change significantly even with small variations in the placement.

Most AEDs have visual guides for pad placement that rely on placing the single pad electrodes at specific locations, such as anterior-anterior locations on an adult's chest. However, this manual approach to proper pad placement is time-consuming and undesirable given that a key priority is to minimise delays to shock delivery. Even with training, electrode placements of conventional AEDs are known to be error prone.

Moreover, one of the key requirements for AEDs is a compact device form factor with small pad footprints for electrode layout and packaging. At the same time, the pads should be capable of acquiring ECG signals with high quality, and delivering defibrillation shocks with high efficacy.

A good design solution for AEDs should offer an optimal trade-off between small device size, and high defibrillation efficacy and high signal quality. Yet, finding such optimal trade-offs for AEDs with a single electrode pair is challenging due to the complex interplay of product requirements and regulatory standards.

In view of this background, there is an unmet need for AEDs having defibrillation pads with improved electrode configurations for electrical measurement and stimulation of patients' hearts.

### SUMMARY

According to the present invention, there is provided an AED, comprising:
two pads to be placed on a patient;
multiple electrode pairs provided on or between the two pads to perform multiple functions of electrical measurement and stimulation of the patient's heart;
a switching circuit and a shock generation circuit connected to the multiple electrode pairs; and
a controller connected to the switching circuit and the shock generation circuit, wherein the controller is configured to perform spatial division multiplexing (SDM) by rapidly switching between the multiple electrode pairs to automatically perform the multiple functions of electrical measurement and stimulation of the patient's heart using the multiple electrode pairs in multiple directions.

The multiple functions of electrical measurement and stimulation of the patient's heart performed by the multiple electrode pairs in multiple directions may comprise:
measuring cardiac electrical signals to detect locations of the two pads;
measuring ECG signals to detect shockable cardiac rhythms; and
delivering doses of defibrillation shocks based on the detected locations of the two pads when shockable cardiac rhythms are detected.

One of the two pads may have one or more electrodes, and the other may have two or more electrodes.

The measured cardiac electrical signals used to detect locations of the two pads may comprise voltage, current, impedance, or any combination thereof.

The controller may be further configured to analyse the measured cardiac electrical signals to detect locations of the two pads based on signal strengths of the measured cardiac electrical signals in multiple directions between multiple electrode pairs.

The detected locations of the two pads may comprise locations of the two pads relative to one another and ventricles of the patient's heart.

The controller may be further configured to control the doses of defibrillation shocks delivered by the two pads based on their detected locations.

The controller may be further configured to rapidly switch between the multiple electrode pairs and automatically select optimal electrode pairs to perform the multiple functions after multiple electrical circuit paths between the multiple electrode pairs have been traversed in multiple directions.

The controller may be further configured to rapidly switch between the multiple electrode pairs so that any combination of the multiple electrode pairs is used to perform any combination of the multiple functions using any combination of the multiple electrical circuit paths between the multiple electrode pairs in any combination of multiple directions.

The present invention also provides a method of using an AED comprising two pads to be placed on a patient, the method comprising:
providing multiple electrode pairs on or between the two pads to perform multiple functions of electrical measurement and stimulation of the patient's heart;
rapidly switching between the multiple electrode pairs to automatically perform the multiple functions of electrical measurement and stimulation of the patient's heart using the multiple electrode pairs in multiple directions.

### BRIEF DESCRIPTION OF DRAWINGS

Embodiments of the invention will now be described by way of example only with reference to the accompanying drawings, in which:

FIG. 1 is a top perspective view of an AED according to an embodiment of the present invention;

FIGS. 2 to 8 are bottom plan views of defibrillation pads of the AED with different multiple-electrode configurations; and

FIGS. 9 to 12 are schematic diagrams of AEDs having defibrillation pads with different multiple, multifunctional electrode configurations in use on adult patients.

### DESCRIPTION OF EMBODIMENTS

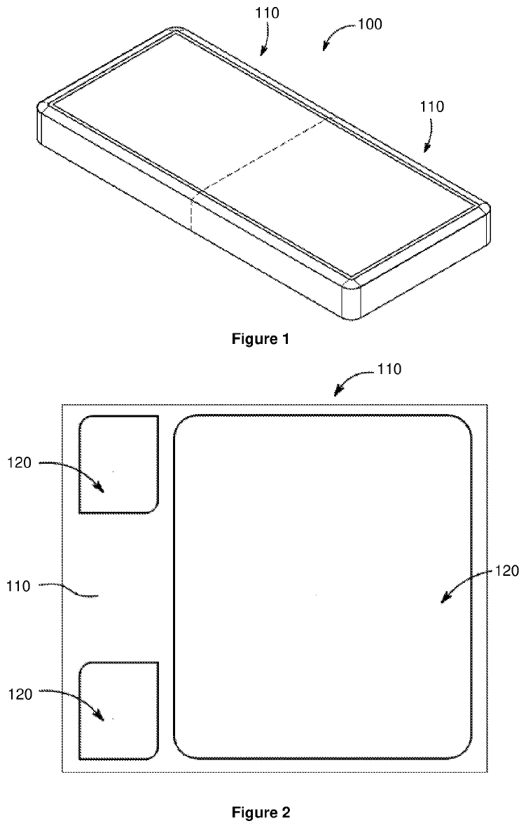

Referring to FIG. 1, an AED 100 according to an embodiment of the present invention may generally comprise two defibrillation pads 110 intended to be separated from one another and placed on a patient, for example in anterior-anterior locations for adults. The AED 100 may have a compact device form factor with small pad footprints. A suitable compact AED 100 is described in further detail in the present applicant's WO 2018/232450 which is hereby incorporated by reference in its entirety.

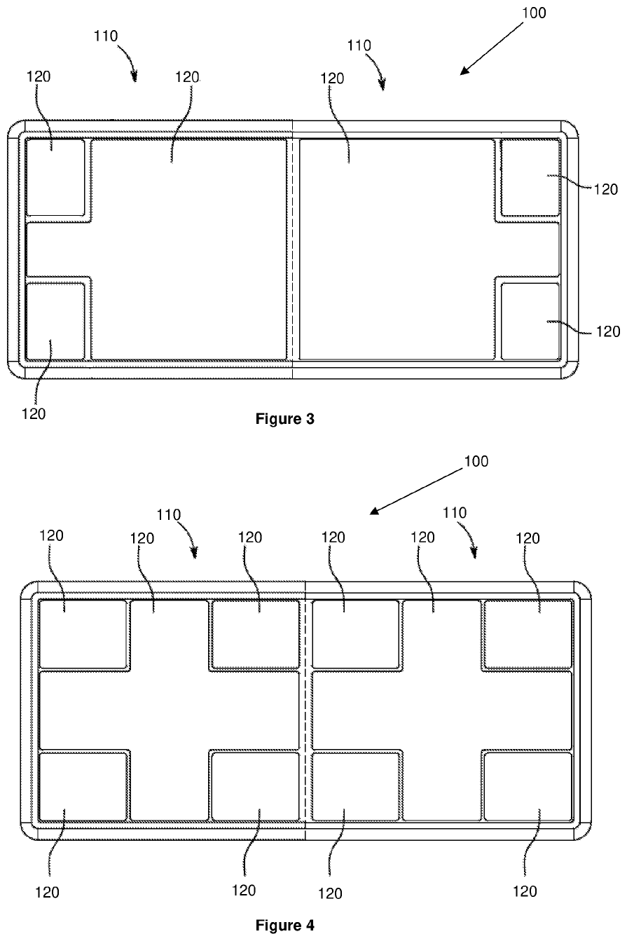

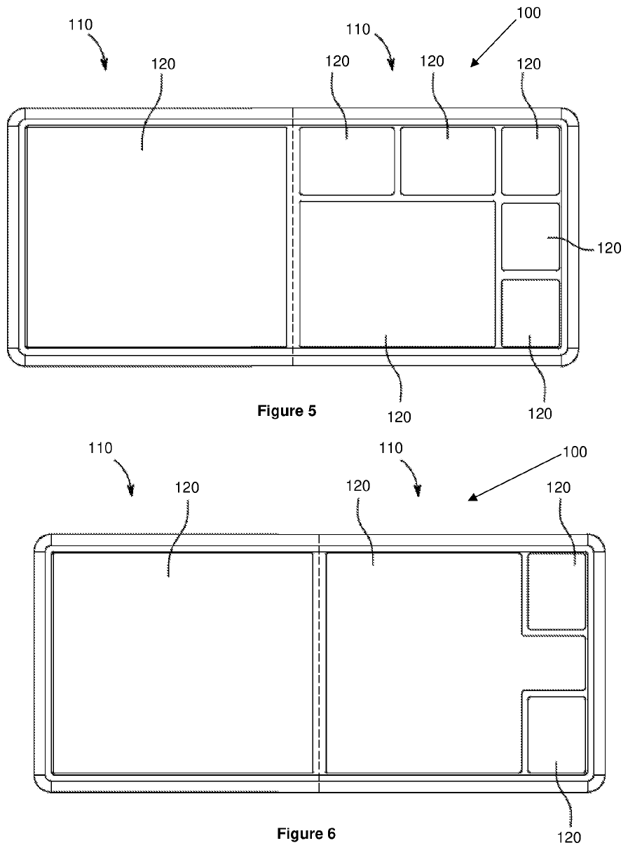

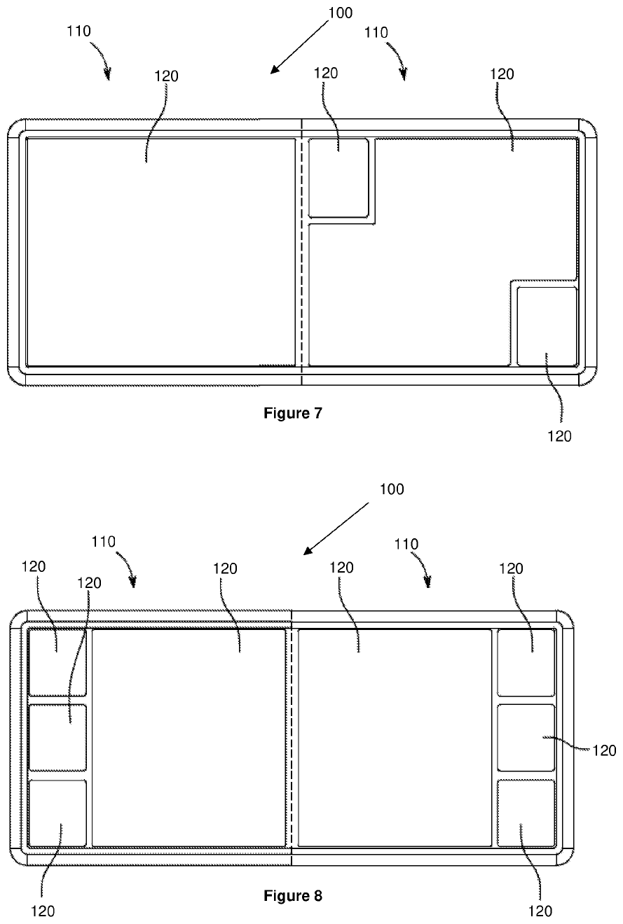

Referring to FIGS. 2 to 8, multiple electrode pairs 120 may be provided on or between the two pads 110 to perform multiple functions of electrical measurement and stimulation of the patient's heart. The multiple electrode pairs 120 may comprise pairs of electrodes 120 collocated on the same one of the two pads 110, and/or pairs of electrodes 120 defined across or between the two pads 110. For example, one of the two pads 110 may have one or more electrodes 120, and the other may have two or more electrodes 120. The two pads 110 may have the same or different number of electrodes 120. The electrodes 120 may have the same or different shapes, sizes, and surface areas. The electrodes 120 may be arranged on the two pads 110 in two subarrays of electrodes 120—one subarray per pad—having the same or different regular, repeating, and uniform patterns or layouts of electrodes 120.

The sizes, spacing, surface area, placement, electrical isolation, packaging, layout, etc of the electrodes 120 on the two pads 110 may depend on the available footprint space, and other design parameters, product requirements and regulatory standards, such as minimum electrode surface areas, available electrical power, cost, etc.

An electronics module (not shown) may be packaged in the enclosures of one or both of the two pads 110. The electronics module may comprise a switching circuit and a shock generation circuit connected to the multiple electrode pairs 120. The electronics module may further comprise a controller, such as one or more processors, connected to the switching circuit and the shock generation circuit.

The electronics module may further comprise other electronic components, such as one or more batteries, which are also packaged the enclosures of one or both of the two pads 110. The electronic components of the AED 100 are described in further detail in the present applicant's WO 2018/232450 referred to above.

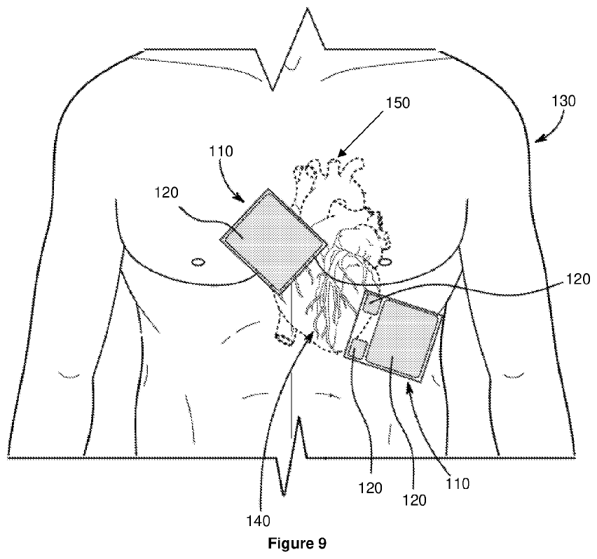

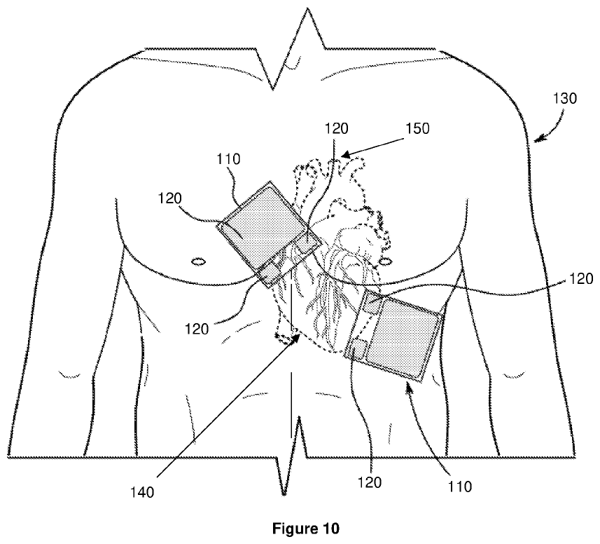

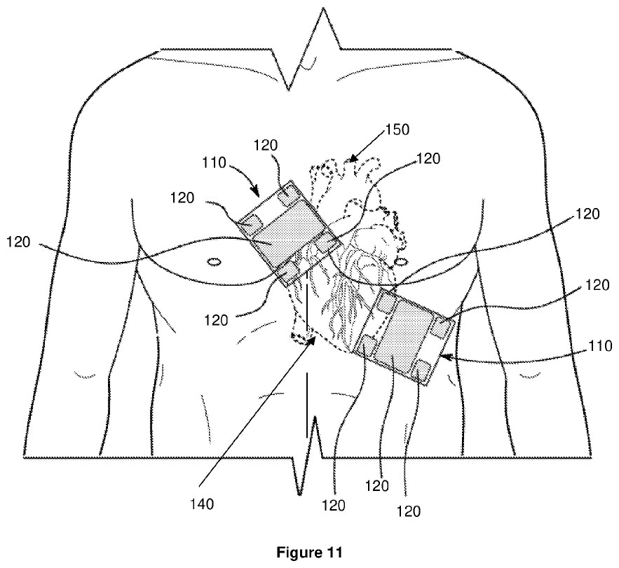

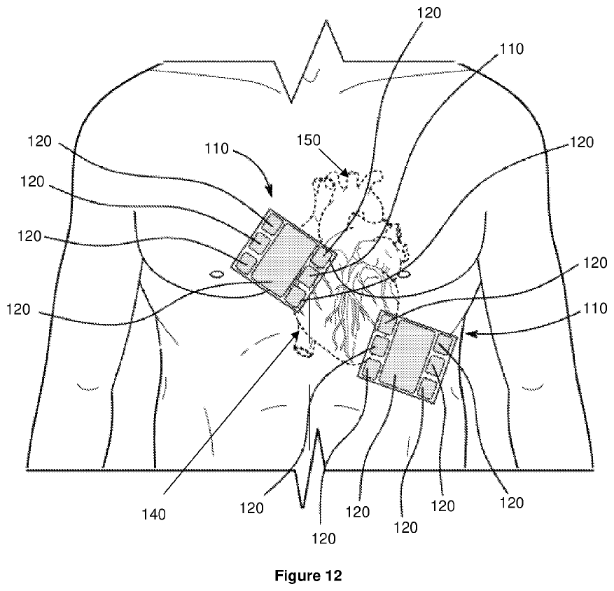

Referring to FIGS. 9 to 12, the two pads 110 may be connected to one another and the electronics module by a lead (not shown) when separated and placed on a patient 130. In use, the controller may be configured to perform SDM by rapidly switching between the multiple electrode pairs 120 to automatically perform the multiple functions of electrical measurement and stimulation of the patient's heart 140 using the multiple electrode pairs 120 in multiple directions.

The multiple functions of electrical measurement and stimulation of the patient's heart 140 performed by the multiple electrode pairs 120 in multiple directions may comprise: measuring cardiac electrical signals to detect locations of the two pads 110; measuring ECG signals to detect shockable cardiac rhythms; and delivering doses of defibrillation shocks by the two pads 110 based on their detected locations when shockable cardiac rhythms are detected.

The multiple electrical measurement functions may be performed by the multiple electrode pairs 120 sequentially or continuously. For example, the multiple electrode pairs 120 may be used to measure cardiac electrical signals and ECG signals before and after doses of defibrillation shocks are delivered.

In some embodiments, each electrode pair 120 may have a predetermined specific functionality, for example, one electrode pair 120 may be used to measure cardiac electrical signals to detect locations of the two pads 110, while another electrode pair 120 may be used to deliver doses of defibrillation shocks by the two pads 110 based on their detected locations. Further or alternatively, each electrode pair 120 may perform some or all of the multiple functions described above sequentially or continuously. For example, the same electrode pairs 120 may be used to measure ECG signals and deliver doses of defibrillation shocks in an alternating sequence.

In other embodiments, each electrode pair 120 may not have a predetermined specific functionality. Instead, the controller may be configured to switch between the multiple electrode pairs 120 and automatically select optimal electrode pairs 120 to perform the multiple functions after some or all multiple point-to-point electrical circuit paths between the multiple electrode pairs have been traversed in multiple directions. In this context, “space division multiplexing” (SDM) may mean any combination of the multiple electrode pairs 120 may be used to perform any combination of the multiple functions using any combination of the multiple point-to-point electrical circuit paths between the multiple electrode pairs 120 in any combination of multiple directions or polarities.

The controller may automatically select optimal electrode pairs 120 to perform the multiple functions based on the measured cardiac signals and the detected locations of the two pads 110. In other words, the controller may automatically select individual electrodes 120 on or between the two pads 110 respectively as optimal electrode pairs 120 as start points and end points respectively of optimal point-to-point circuits between the two pads 110 for performing the multiple functions.

For example, the optimal location for delivering a first phase of a biphasic defibrillation shock to an adult is for an electrode 120 closest to the patient's right ventricle to be assigned a negative polarity. To optimise defibrillation efficacy, the controller may detect which of the two pads 110 has a location closest to the patient's right ventricle, and automatically select one electrode 120 of an electrode pair 120 on that pad 110 as a start point to have a negative polarity for delivering the first phase of a biphasic defibrillation shock to a paired second electrode 120 of the electrode pair 120 on the other pad 110 which acts an end point for the circuit path.

The measured cardiac electrical signals used to detect locations of the two pads 110 may comprise voltage, current, impedance or any combination thereof. The detected locations of the two pads 110 may comprise locations and orientations of the two pads 110 relative to one another and ventricles 150 of the patient's heart 130.

For example, as shown in FIGS. 9 to 12, the detected locations and orientations of the two pads 100 with respect to one another and the ventricles 150 may comprise anterior-anterior locations on an adult patient with one pad 110 on the upper right side of the chest and the other pad 110 on the lower left side of the chest. For child patients (not shown), the detected locations and orientations of the two pads 100 with respect to one another and the ventricles 150 may comprise anterior-posterior locations with one pad 110 on the front of the chest and the other pad 110 on the back of the chest.

In some embodiments, the controller may be configured to detect if a patient is an adult or a child based on the detected locations and orientations of the two pads 110 with respect to one another and the ventricles 150. The controller may be further configured to automatically select an adult dose or a child dose of defibrillation shocks based, at least in part, on the detected locations and orientations of the two pads 110 with respect to one another and the ventricles 150.

The controller may be configured to analyse the measured cardiac electrical signals to detect locations and orientations of the two pads 110 based on signal strengths of the measured cardiac electrical signals in multiple directions between multiple electrode pairs 120.

For example, the highest signal strengths of measured cardiac voltage between the multiple electrode pairs 120 in multiple directions may be used to detect which of the two pads 110 is closest to the patient's ventricles 150, or whether the two pads 120 are located or oriented on a mean cardiac electrical axis (not shown) of the patient 130 which extends down to the left from the ventricles 150.

The controller may be further configured to control the doses (or strength and direction) of defibrillation shocks delivered by the two pads 110 based on their detected locations and orientations. The doses of defibrillation shocks may be controlled by varying their polarity and energy. The energy of the doses of defibrillation shocks may be controlled by varying their voltage, current, waveform, time duration, or any combinations thereof. The defibrillation shocks may comprise monophasic or biphasic shocks.

The SDM of the multiple electrode pairs 120 performed by the controller may allow the doses of defibrillation shocks to be focused on the mean cardiac electrical axis of the patient 130. In turn, this may allow the energy levels of the doses of defibrillation shocks to be lowered, and the surface areas of individual electrodes 120 to be smaller, without reducing defibrillation efficacy. This may reduce the power requirements, battery size, form factor, and cost of the AED 100, or extend its shelf life or service life.

Furthermore, the SDM of the multiple electrode pairs 120 performed by the controller may allow cardiac electrical signals and ECG signals to be measured using electrodes 120 having a smaller surface area without reducing signal quality.

Embodiments of the present invention may therefore provide AEDs 100 that offer improved trade-offs between small device size, and high defibrillation efficacy and high cardiac signal quality.

Embodiments of the present invention provide AEDs having defibrillation pads with multiple, multifunctional electrode pairs that are both generally and specifically useful for performing multiple functions of electrical measurement and stimulation of patients' hearts.

Unless the context requires otherwise, the word “comprising” means “including but not limited to,” and the word “comprises” has a corresponding meaning.

The scope of the invention enabled and supported by the above examples is defined by the claims that follow.

1. An automated external defibrillator (AED), comprising:
two pads to be placed on a patient;
multiple electrode pairs provided on or between the two pads to perform multiple functions of electrical measurement and stimulation of the patient's heart;
a switching circuit and a shock generation circuit connected to the multiple electrode pairs; and
a controller connected to the switching circuit and the shock generation circuit, wherein the controller is configured to perform spatial division multiplexing by rapidly switching between the multiple electrode pairs to automatically perform the multiple functions of electrical measurement and stimulation of the patient's heart using the multiple electrode pairs in multiple directions.

2. The AED of claim 1, wherein the multiple functions of electrical measurement and stimulation of the patient's heart performed by the multiple electrode pairs in multiple directions comprise:
measuring cardiac electrical signals to detect locations of the two pads;
measuring ECG signals to detect shockable cardiac rhythms; and
delivering doses of defibrillation shocks by the two pads based on their detected locations when shockable cardiac rhythms are detected.

3. The AED of claim 1, wherein one of the two pads has one or more electrodes, and the other has two or more electrodes.

4. The AED of claim 2, wherein the measured cardiac electrical signals used to detect locations of the two pads comprise voltage, current, impedance, or any combination thereof.

5. The AED of claim 2, wherein the controller is further configured to analyse the measured cardiac electrical signals to detect locations of the two pads based on signal strengths of the measured cardiac electrical signals in multiple directions between multiple electrode pairs.

6. The AED of claim 2, wherein the detected locations of the two pads comprise locations of the two pads relative to one another and ventricles of the patient's heart.

7. The AED of claim 2, wherein the controller is further configured to control the doses of defibrillation shocks delivered by the two pads based on their detected locations.

8. The AED of claim 1, wherein the controller is further configured to rapidly switch between the multiple electrode pairs and automatically select optimal electrode pairs to perform the multiple functions after multiple point-to-point electrical circuit paths between the multiple electrode pairs have been traversed in multiple directions.

9. The AED of claim 8, wherein the controller is further configured to rapidly switch between the multiple electrode pairs so that any combination of the multiple electrode pairs is used to perform any combination of the multiple functions using any combination of the multiple point-to-point electrical circuit paths between the multiple electrode pairs in any combination of multiple directions.

10. A method of using an AED having two pads to be placed on a patient, the method comprising:
providing multiple electrode pairs on or between the two pads to perform multiple functions of electrical measurement and stimulation of the patient's heart;
rapidly switching between the multiple electrode pairs to automatically perform the multiple functions of electrical measurement and stimulation of the patient's heart using the multiple electrode pairs in multiple directions.
